(19) United States
(12) Patent Application Publication
(10) Pub. No.: US 2025/0114492 A1
(43) Pub. Date: Apr. 10, 2025

(54) Disinfection System For Wheelchairs

(71) Applicant: United States Government As Represented By The Department Of Veterans Affairs, Washington, DC (US)

(72) Inventor: Kristin Mate, Biloxi, MS (US)

(21) Appl. No.: 18/907,722

(22) Filed: Oct. 7, 2024

Related U.S. Application Data
(60) Provisional application No. 63/588,155, filed on Oct. 5, 2023.

(51) Int. Cl. A61L 2/10; A61G 12/00; A61L 2/24; A61L 2/26; G08B 21/18

(52) U.S. Cl. CPC A61L 2/10; A61G 12/00; A61L 2/24; A61L 2/26; A61L 2202/11; A61L 2202/121; A61L 2202/122; A61L 2202/14; A61L 2202/16; G08B 21/18

(57) ABSTRACT

A disinfection system for a wheelchair may include an enclosure having a front wall, a rear wall, a top wall extending between a top edge of the front wall and a top edge of the rear wall, and an entry door pivotably secured to a side edge of either the front wall or the rear wall. At least one disinfection lamp is disposed within the enclosure and configured to disinfect the surface of the wheelchair stored therein (optionally, with UV-C light).

### CROSS-REFERENCE TO RELATED APPLICATIONS

The application claims priority to and the benefit of the filing date of U.S. Provisional Patent Application No. 63/588,155, filed Oct. 5, 2023, the entirety of which, including the appendices thereof, is hereby incorporated by reference herein.

### FIELD

The application is generally related to wheelchairs and, more particularly, to a system and method of use thereof for disinfecting, storing, and tracking public use wheelchairs.

### BACKGROUND

Facilities such as hospitals, rehabilitation centers, airports, tourist attractions, and the like often have wheelchairs available for use by the general public. Typically, the wheelchairs are obtained from a storage area by a user upon arrival at the facility, utilized while at the facility, and returned to the storage area when the user leaves the facility. As such, at any given time, the wheelchairs can be located at multiple locations in the facility or in proximity to the facility, and are often left where convenient for the user when departing, such as in a parking lot, rather than in the proper storage area.

Due to high usage by multiple persons and exposure to the elements, public use wheelchairs may become damaged or soiled fairly quickly when compared to privately owned wheelchairs. Additionally, public use wheelchairs have the potential to spread germs amongst users as they are rarely sanitized between uses.

From the foregoing, one recognizes the need for systems and methods that sanitize, store, and/or track the location of public use wheelchairs that are provided at a given facility.

### SUMMARY

Embodiments of the disclosed disinfection system for a wheelchair are disclosed.

In one aspect, the disinfection system comprises an enclosure configured to receive the wheelchair therein and at least one disinfection lamp disposed within the enclosure. The at least one disinfection lamp can be configured to disinfect the surfaces of the wheelchair stored therein (optionally, with germicidal ultraviolet-C (UV-C) light). In some aspects, the enclosure comprises a front wall, a rear wall, a top wall extending between the front wall and the rear wall, and an entry door pivotably secured to one of the front wall and the rear wall.

In another aspect, a wheelchair tracking system comprises a plurality of enclosures configured to receive a wheelchair and at least one wheelchair. Each wheelchair is received within a respective enclosure of the plurality of enclosures. At least one computing device is configured to cause one enclosure of the plurality of enclosures having a wheelchair therein to release the wheelchair upon receipt of an identifier of a user.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 is a perspective view of a disinfection system for a wheelchair in accordance with an embodiment of the present disclosure.

FIG. 2 is a right side plan view the disinfection system for a wheelchair shown in FIG. 1.

FIG. 3 is a rear plan view of the disinfection system for a wheelchair shown in FIG. 1.

FIG. 4 is a first plan view of the disinfection system for a wheelchair shown in FIG. 1.

FIG. 5 is a perspective view of a disinfection system for a wheelchair in accordance with an alternate embodiment of the present disclosure.

FIG. 6 is a left side plan view of the disinfection system for a wheelchair shown in FIG. 5.

FIG. 7 is a first plan view of the disinfection system for a wheelchair shown in FIG. 5.

FIG. 8 is a block diagram of a computing device for implementing the methods disclosed herein, in accordance with some embodiments of the present invention.

FIG. 9 is a perspective view of an exemplary kiosk system with a plurality of enclosures for housing respective wheelchairs.

FIG. 10 is a block diagram of a system for tracking wheelchairs of a plurality of kiosks.

FIG. 11 is a schematic diagram of an exemplary kiosk station as disclosed herein.

FIG. 12 is an exemplary kiosk station as disclosed herein.

FIG. 13 is a perspective view of an exemplary carousel of a kiosk station.

FIG. 14 is a perspective view of an exemplary wheelchair handling assembly for use with the carousel of the kiosk station.

FIG. 15 is a schematic diagram of an exemplary kiosk station as disclosed herein.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings.

Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” can optionally include plural referents unless the context clearly dictates otherwise. For example, use of the term “a loop” can represent the disclosure of embodiments in which only a single loop is provided, as well as the disclosure of embodiments in which a plurality of such loops are provided. Thus, it is understood that the terms “a,” “an,” and “the” can refer to one or more of the stated elements.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

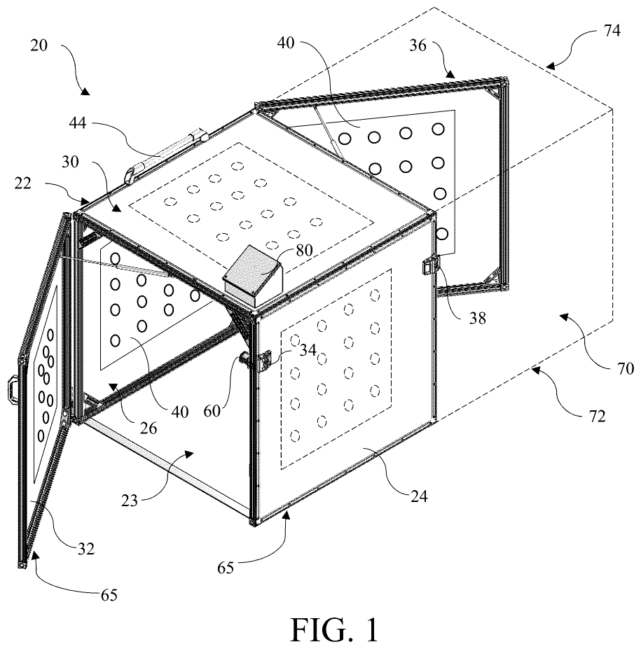

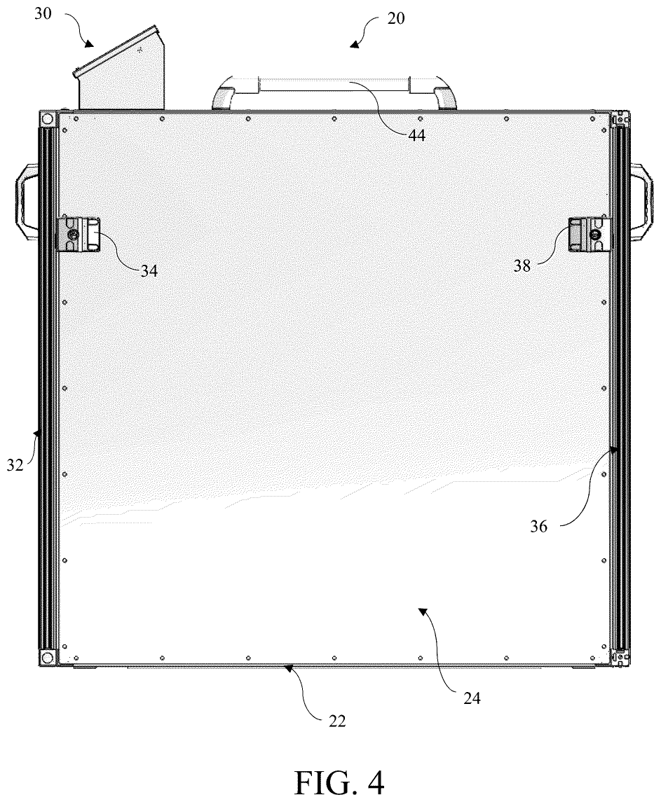

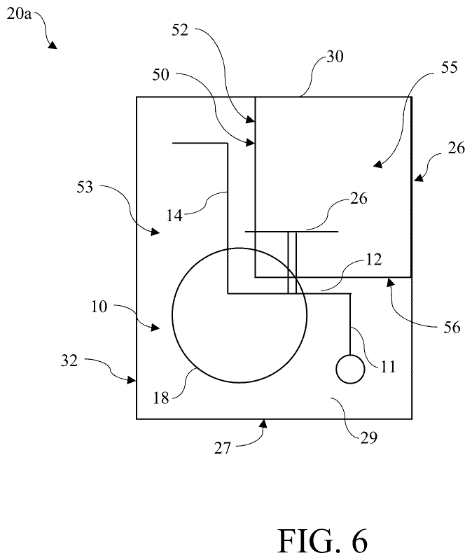

Referring now to FIGS. 1-6, a disinfection kiosk system 20 for wheelchairs 10 (see FIG. 6) in accordance with an embodiment of the present disclosure is shown. In some aspects, the disinfection kiosk system 20 can include an enclosure 22 that is configured to receive a wheelchair 10 therein, and at least one disinfection lamp 40 (optionally, a plurality of disinfection lamps) that is configured to disinfect the wheelchair 10 by flooding its surfaces with germicidal light (e.g., germicidal ultraviolet light). Specifically, germicidal ultraviolet-C (UV-C) light within the 200-280 nm wavelength range can be used. However, it is contemplated that other frequency ranges that provide a germicidal effect can be used. In some aspects, the disinfection lamps can comprise xenon bulbs. In some aspects, the disinfection lamps can be configured to effectively disinfect within a predetermined time, such as, for example, less than five minutes, less than four minutes, less than three minutes, less than two minutes, or less than one minute. In some aspects, the predetermined time can be selected to achieve a desired disinfection level, such as >95%, >96%, >97%, >98%, or >99% sporicidal disinfection.

As shown, the enclosure 22 includes a front wall 24, a rear wall 26 (that is optionally parallel or substantially parallel to the front wall 24), and a top wall 30 that extends between the top edges of the front wall 24 and the rear wall 26. An entry door 32 is pivotably attached to a side edge of the rear wall 26 by hinges 33 and provides access to the interior volume 23 of the enclosure 22. An exit door 36 is similarly secured to the other side edge of the rear wall 26 by hinges 39 and is configured to allow easy retrieval of the wheelchair 10 from the enclosure 22 after the wheelchair has been disinfected. In some aspects, locks 34 and 38 are provided for both the entry door 32 and the exit door 36, respectively, to ensure that personnel may not access the enclosure 22 during the disinfection process in which the disinfection lamps 40 are activated, as discussed in greater detail below. Note, the inside surfaces of the disinfection kiosk 20 can be highly reflective to reflect the UV-C light onto as many surfaces of the wheelchair being disinfected as possible. Preferably, the walls of the kiosk 20 are easily disassembled for easy storage but also easily reassembled following storage. Additionally, some embodiments may include wall portions that are pivotably secured to each other so that the kiosk 20 is foldable, with braces being used to add rigidity to the disinfection kiosk 20 when in use.

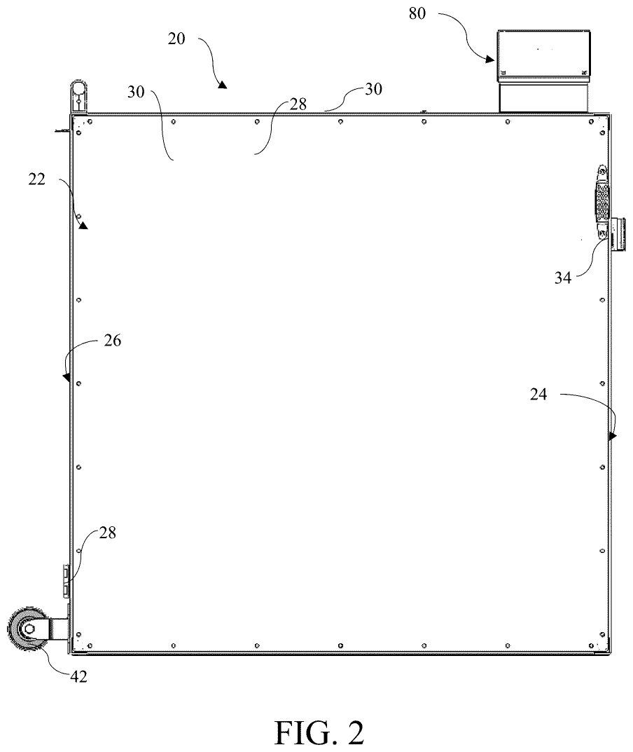

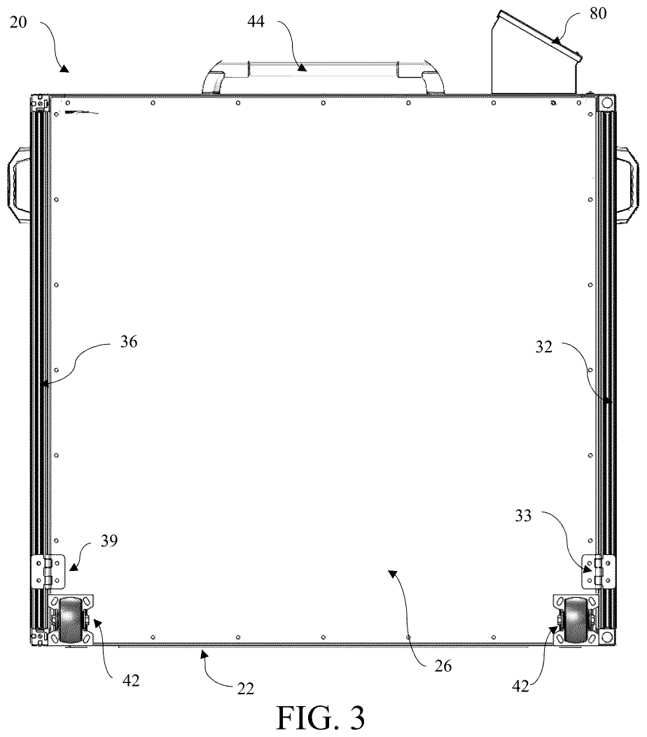

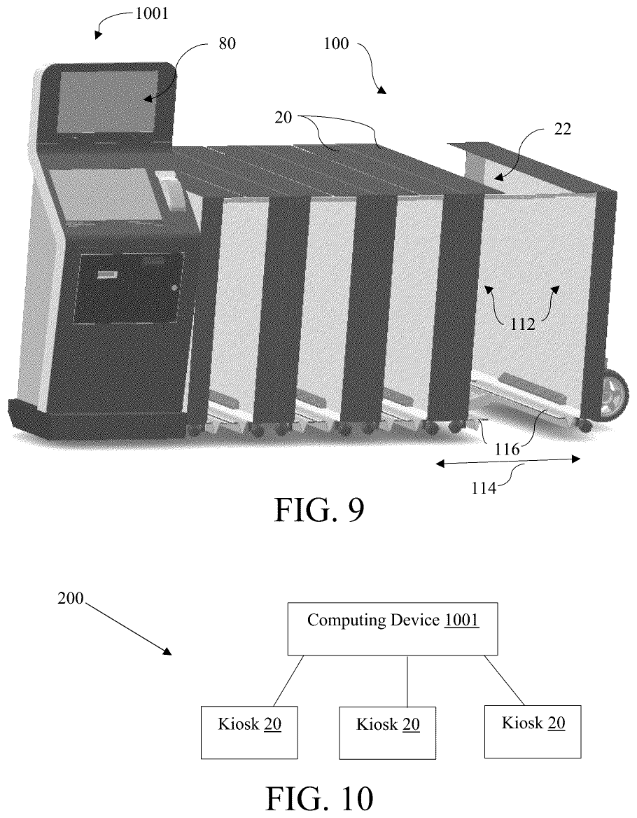

As best seen in FIGS. 2 and 3, the disinfection kiosk 20 can include at least a pair of wheels 42 secured along the bottom edge of the rear wall 26 and a handle assembly 44 secured along the top edge of the rear wall 26. The handle assembly 44 and wheels 42 allow for a user to readily move the disinfection kiosk 20 to a desired location by simply tipping the kiosk 20 rearwardly and pulling it along by the handle 44. Additionally, the disinfection kiosk 20 includes a data input device 80, which can optionally be disposed on the top wall 30 adjacent the entry door 32. In other aspects, the data input device 80 can be external to or independent of the enclosure 22, as shown in FIG. 9. The data input device 80 may include a touch pad, a scanner, a card reader, and/or other structure that receives an input from a user, and the data input device may be used to input data by a user as discussed in greater detail below. As shown in FIG. 1, the disinfection kiosk system 20 may also include a corral 70 that is configured to store a plurality of wheelchairs 10 after they have been disinfected. Preferably, the corral 70 is disposed adjacent or in proximity to the exit door 36 of the disinfection kiosk 20. In exemplary aspects, the corral 70 is formed by a fence 72 that includes a gate 74 at its distal end. Note, in some alternate embodiments, various edges of the top wall, bottom wall, side walls, and doors may be connected by hinges. The hinges allow the kiosk system to be foldable for easier storage and facilitate both set-up and take-down of the unit. Braces may be used between adjacent walls to lend rigidity to the kiosk.

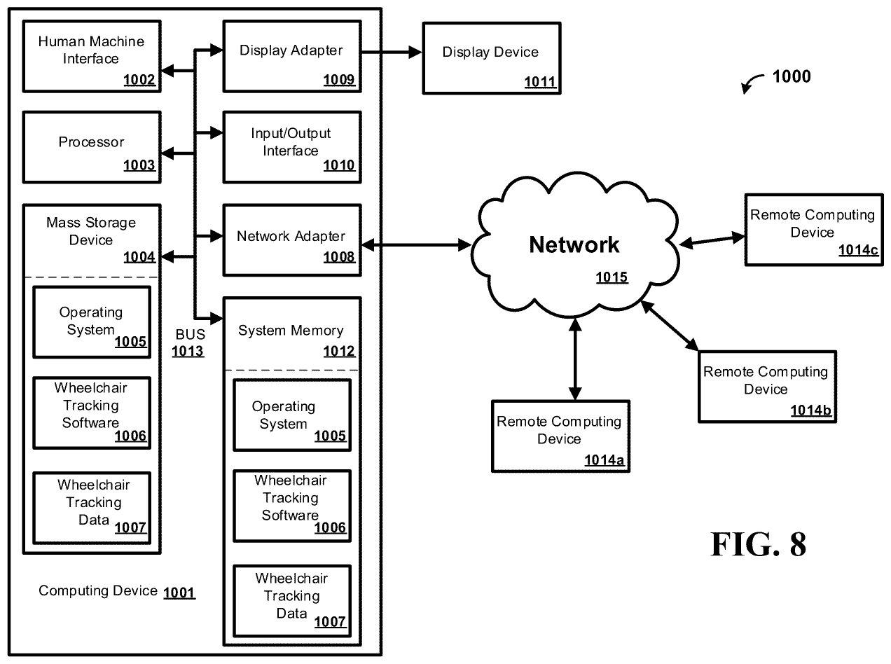

An example usage of the disinfection kiosk system 20 may include an individual (e.g., caregiver/staff member, or patient) that approaches the disinfection kiosk 20 after having used a wheelchair 10. The individual may then use the data input device 80 to either input an access code, a phone number, or other identifying information via a touch pad, and/or scan an identification card, thereby unlocking the entry door 32. The individual then pushes the wheelchair 10 into the enclosure 22 and closes the entry door 32. The wheelchair 10 can include an identifier (optionally, a scannable identifier, such as a barcode, QR code, etc.), that may be used for tracking both the location of the wheelchair and its cleanliness status. Internal rails (not shown) can be provided on the inner surfaces of the front wall 24 and the rear wall 26 to prevent contact of the wheelchair 10 with inside surfaces of the kiosk 20. A sensor 60 (e.g., a contact sensor) is provided on the enclosure 22 so that the sensor 60 can detect when the entry door 32 is closed and the enclosure 22 is secure. The user next initiates the disinfection process by energizing the disinfection lamp(s) 40. Alternatively, the disinfection process may be initiated automatically when the user closes the entry door 32. After a predetermined amount of time, the disinfection lamps 40 are de-energized, at which point the lock 38 on the exit door 36 is automatically disengaged, allowing the individual to open the exit door 36 and roll the wheelchair 10 out of the enclosure 22 and then into the corral 70 for the next usage. Preferably, the exit door 36 also includes a sensor or switch (not shown), for example a contact sensor, that produces a signal or alarm that is indicative of the exit door 36 being in the open position. The sensor(s) configured to detect the entry and exit doors being in open positions can provide the signal to a computing device 1001 (FIG. 8) controlling operation of the disinfection lamps 40. The computing device 1001 can be de-energized upon receiving a signal from the sensor(s), thereby providing an interlock that inhibits energizing of the disinfection lamps 40 unless the door(s) (e.g., entry door 32) and exit door 36) are closed.

Optionally the door(s) of the disinfection kiosk 20 can be retained in the closed position with magnets. Each door can be provide with a sensor that is operable to de-energize the disinfection lamps 40 upon sensing that the respective door is in an open position.

In some aspects, a motion sensor (not shown) can be disposed within the interior volume 23 of the enclosure 22 and can be configured to prevent energizing the disinfection lamps 40 if the sensor produces a signal or other output indicative of motion being detected inside the interior volume 23 of the enclosure 22. Additionally, if motion is detected after the disinfection process has commenced, delivery of power to the disinfection lamps 40 can be automatically stopped, and the locks 34 and 38 on the entry door and the exit door 32 and 36 can be disengaged. Additionally, as best seen in FIG. 1, the enclosure 22 can have an open bottom to allow easy egress for anybody who may become trapped therein. For example, the bottom can be defined by bottom edges 65 of each of the front wall, the rear wall, the entry door, and the exit door. The walls and doors of the enclosure 22 can be formed of stainless steel, aluminum, or similar material, limiting the weight of the enclosure. If an individual becomes trapped in the enclosure 22, the individual may simply lift up on the enclosure and exit through the open bottom end. Note, the data input device 80 also allows an individual to input information related to the specific wheelchair 10 that the individual has used, so that any damage to the wheelchair or unsafe conditions concerning the wheelchair may be recorded and transferred to the proper authority. This information may be transferred via wireless communications to a remote station so that the number, condition, locations, etc., of the wheelchairs may be monitored remotely from the kiosk locations. In further aspects, the information can be stored on the kiosk. Stored information can later be retrieved, for example, by downloading via wired or wireless connection or recalling the information for display on the data input device 80.

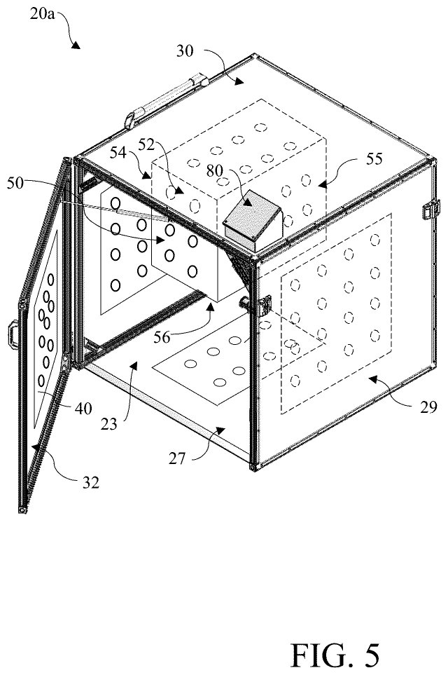

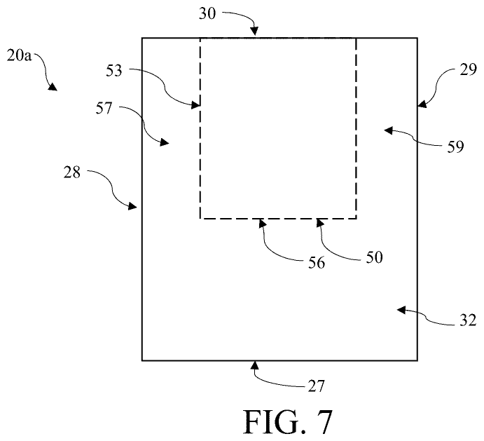

The embodiment of the disinfection kiosk system 20a shown in FIGS. 5-7 differs primarily from the previously discussed embodiment in that the disinfection kiosk 20a includes a housing 50 disposed within the interior volume 23 of the enclosure 22. As shown, the housing 50 may optionally be rectangular or substantially rectangular. The housing 50 includes a front wall 52, a first side wall 54 and a second side wall 55 that are substantially parallel and extend from the side edges of the front wall 52, and a bottom wall 56 that extends between the bottom edges of the first side wall 54 and the second side wall 55. The housing 50 extends downwardly from an inner surface of the top wall 30 of the enclosure 22 and extends to the inner surface of the rear wall 26 of the enclosure 22.

As best seen in FIG. 6, the front wall 52 of the housing 50 defines a gap 53 between the inner surface of the entry door 32, and the first side wall 54 and the second side wall 55 form gaps 57 and 59, respectively, with the inner surfaces of the first side wall 28 and the second side wall 29 of the enclosure 22. The gap 53 defined between the front wall 52 of the housing and the entry door 32 is configured to receive the back rest 14 portion of the wheelchair 10 therein, whereas the gaps 57 and 59 defined between the first and the second side walls 54 and 55 of the housing 50 and the side walls 28 and 29 of the enclosure 22, respectively, are configured to receive the arm rests 16 of the wheelchair 10 therein. Additionally, the height of the housing 50 is selected such that the bottom wall 56 of the housing 50 is disclosed in close proximity to the seating portion 12 of a standard wheelchair, as shown in FIG. 6. Optionally, as shown in FIG. 5, the front wall 52, the first and second side walls 54 and 55, and the bottom wall 56 of the housing may each include disinfection lamps 40 disposed thereon. As such, the housing 50 is configured to position disinfection lamps 40 in close proximity to the surfaces of the backrest 14, seating portion 12, and armrests 16 of the wheelchair that is being disinfected. The inner surfaces of the entry door 32 and the sidewalls 28 and 29 may also include disinfection lamps 40 in order to disinfect the remaining frame 11 and wheels 18 of the wheelchair 10. Note also, unlike the previously discussed embodiment of the kiosk system, the enclosure 22 of the present embodiment may include a bottom wall 27 on which disinfection lamps 40 may also be placed.

In some aspects, the kiosk 20 can have a single chamber that is configured to hold one wheelchair at a time. Optionally, in these aspects, the kiosk 20 can be a stand-alone device.

Referring to FIG. 9, a kiosk station 100 can comprise an input device 80 and a plurality of kiosks 20. In some aspects, the kiosk station 100 can comprise at least two kiosks 20, at least four kiosks, at least six kiosks, at least eight kiosks, or at least ten kiosks.

In some aspects, each enclosure 22 can be collapsible. For example, the enclosure 22 can comprise a pair of side walls 112 that are spaced along a horizontal axis 114. The side walls 112 can be coupled to each other to permit movement of the side walls relative to each other about and between a collapsed configuration and an expanded configuration. In this way, the enclosure 22 can be configured to receive a collapsible wheelchair in an expanded configuration during disinfection, and the enclosure 22 and wheelchair can then be collapsed for compact storage. A flexible material (not shown) (e.g., synthetic flashspun high-density polyethylene fibers, such as Tyvek material) can be configured to extend between the side walls to enclose the enclosure 22. In some aspects, the enclosures 22 can comprise lower rails 116 that are configured to receive respective wheels of the wheelchair.

Optionally, the kiosk station 100 can be configured to lock each wheelchair within each enclosure 22 (e.g., using a latch that extends around a portion of the wheelchair). In this way, the kiosk station 100 can inhibit undesired removal of a wheelchair without requiring use of a locked door. In this way, the kiosk station 100 can prevent removal of wheelchairs of enclosures where rigid doors are not provided. This can be advantageous for the enclosures 22 that are collapsible.

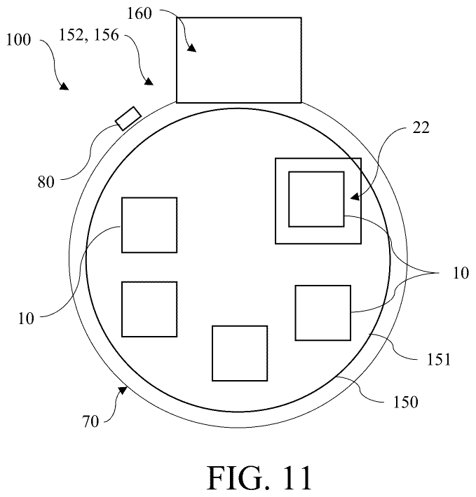

Referring to FIG. 11, in another aspect, a kiosk station 100 can comprise a conveyor 150. For example, the conveyor can be a rotating carousel 151. The conveyor can carry the wheelchair 10 from a receiving area 152 to an enclosure 22 where the wheelchair is disinfected. In some aspects, the wheelchair can be stored on the conveyor until an individual requests a wheelchair at the input device 80. In some aspects, the kiosk station 100 can hold only a single wheelchair. In other aspects, the kiosk station 100 can be configured to store a plurality of wheelchairs.

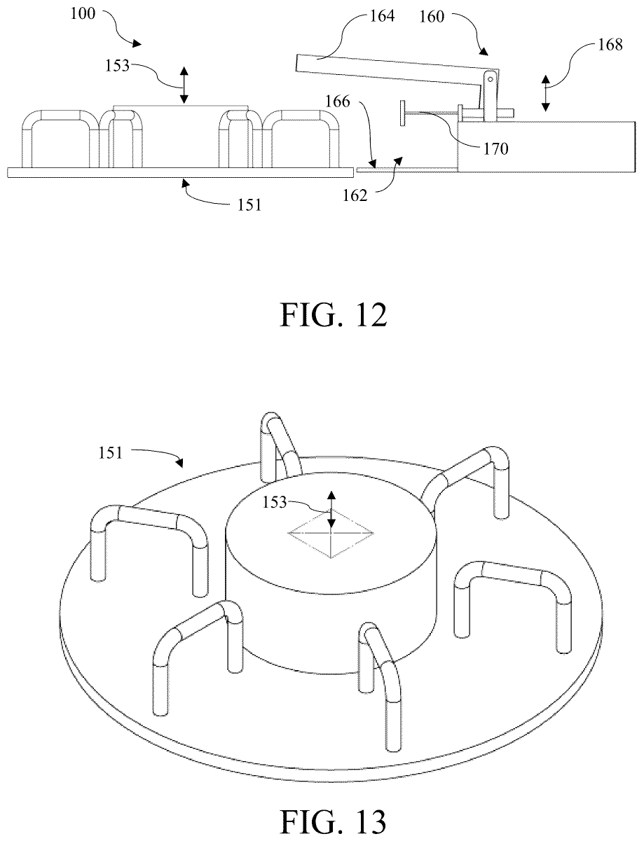

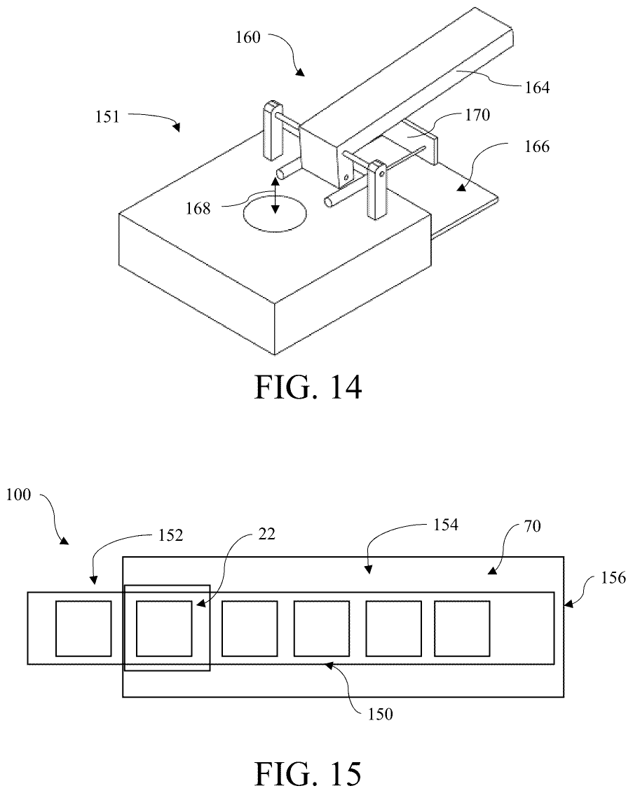

Referring to FIGS. 12-14, the kiosk station 100 can comprise a rotating carousel 151 that is rotatable around an axis 153. A loading assembly 160 can be configured to receive a wheelchair at in a loading area 162. The loading assembly 160 can comprise at least one arm 164 that is configured to pivot to press against the wheelchair to secure the wheelchair between a base 166 and the arm 164. Upon securing the wheelchair, the loading assembly can pivot about a pivotal axis 168 to position the wheelchair at the rotating carousel (in the position shown in FIG. 12. The loading assembly 160 can comprise a pusher 170 (e.g., comprising at least one piston) that is configured to move the wheelchair from the loading assembly 160 onto the rotating carousel 151.

In further aspects, and with reference to FIG. 15, the conveyor 150 can be a linear conveyor (e.g., a belt conveyor or roller conveyor). The conveyor 150 can receive the wheelchair 10 at a receiving area 152, carry the wheelchair to enclosure 22 where the wheelchair is disinfected, and the carry the wheelchair to a storage area 154. Accordingly, in some aspects, the kiosk station 100 can comprise a corral 70 to store a plurality of wheelchairs. The kiosk station 100 can deliver a wheelchair 10 to an individual upon request at a retrieval area 156.

In some aspects, an alert (e.g., an indicator light) can indicate if a wheelchair is working or needs to be serviced.

In another sample usage, a patient, caregiver, staff member, or other individual may approach the kiosk and input in the individual's name, phone number, or other identifier by way of the data input device 80. In response, the kiosk system dispenses a wheelchair from the corral area. Preferably, each wheelchair can include a tracking device such as, but not limited to, RFID, Bluetooth, Air Tags, etc. When the wheelchair has been outside of the kiosk for greater than a predetermined time, the computing system of the kiosk may automatically send out an alert (e.g., a prompt) to the recipient's device (e.g., through a user interface of the recipient's phone, such as an application's user interface displayed on the phone's screen) asking whether or not the wheelchair is still in use. If the user indicates through the user interface of the phone that the wheelchair is still in use, the timer is reset. If the user indicates through the user interface of the phone that the wheelchair is no longer in use or there is no response, the computing system may notify the staff that the wheelchair should be retrieved and returned to the kiosk. When the person returning the wheelchair to the kiosk requests to access the kiosk, the computing system of the kiosk prompts the user regarding the fitness of the wheelchair for further and asks the user if any conditions exist that require attention. The computing system may request this information by way of the data input device 80, or alternately, by sending a message to the phone or computing device of the person returning the wheelchair. The user may also indicate that the wheelchair needs to be taken out of service. If a wheelchair can no longer be tracked, the computing system of the kiosk will alert an associated system at a related facility, such as a repair center, of those wheelchairs that need to be repaired or replaced due to theft, tracker failure, etc.

Still other kiosk systems in accordance with the present invention are automated. For example, upon returning a wheelchair to the kiosk, a user may press a single button of the user input device 80, upon which the computing device causes the doors of the kiosk to close and the disinfection process to start. Upon the completion of the disinfection process, the kiosk system computing system of the kiosk causes the disinfected wheelchair to be conveyed into the corral so that the wheelchair may be safely stored for later use.

Further, although disinfection lamps are disclosed herein for disinfecting the wheelchairs, other disinfection systems are contemplated, such as, for example, a mister configured to deliver atomized chemical disinfectant to the wheelchair.

In some aspects, a wheelchair tracking system 200 can be configured to track a plurality of wheelchairs associated with a particular location (e.g., a facility). For example, optionally, the wheelchair tracking system 100 can comprise at least one computing device 1001 that is configured to communicate with a plurality of kiosks 20. In some aspects, each kiosk can have a help button that is configured to provide an alert to a staff member when assistance is needed.

In some aspects, the computing device 1001 can be configured to log events, such as sanitation occurrences. For example, each sanitation can be configured to store and associate one or more of: a time of receipt of a wheelchair at a kiosk, which kiosk received the wheelchair, an identifier associated with the wheelchair, a time of removing a wheelchair from a kiosk, an identity of the user removing a wheelchair, an identity of the user returning the wheelchair. In this way, if a wheelchair is lost, the log can indicate which individual removed the wheelchair.

In some aspects, the computing device 1001 can be configured to permit an individual to identify a wheelchair that needs service, repair, or other attention.

FIG. 8 shows an exemplary computing system 1000 that can be used to control operation of the disinfection system 20 and wheelchair tracking system 100. In various aspects, the computing system 1000 can be a stand-alone computer, such as a tablet or touchpad. For example, in some aspects, the data input device 80 can be embodied as a portion of a dedicated tablet. In other aspects, the computing system can comprise a plurality of computing devices (e.g., a plurality of kiosk stations 100) that communicate with a central server. In various aspects, the plurality of computing devices can communicate through a cloud-based network. As can be understood, the computing system 1000 can further comprise user's connected devices (e.g., phone, tablet, etc.).

Computing system 1000 can include a computing device 1001 and a display 1011 in electronic communication with the computing device, which can be any conventional computing device, such as, for example and without limitation, a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. In some optional embodiments, a smart phone, tablet, or computer (i.e., a laptop or desktop computer) can comprise both the computing device 1001 and the display 1011. Alternatively, it is contemplated that the display 1011 can be provided as a separate component from the computing device 1001. For example, it is contemplated that the display 1011 can be in wireless communication with the computing device 1001, thereby allowing usage of the display 1011 in a manner consistent with that of the display of the smartphone as disclosed herein.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as wheelchair tracking data 1007 and/or program modules such as operating system 1005 and wheelchair tracking software 1006 that are accessible to and/or are operated on by the one or more processors 1003.

The computing device 1001 may also comprise other removable/non-removable, volatile/non-volatile computer storage media. A mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and the wheelchair tracking software 1006 may be stored on the mass storage device 1004. One or more of the operating system 1005 and the wheelchair tracking control software 1006. Wheelchair tracking data 1007 may also be stored on the mass storage device 1004. The wheelchair tracking 1007 may be stored in any of one or more databases known in the art. The databases may be centralized or distributed across multiple locations within the network 1015.

A user may enter commands and information into the computing device 1001 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 1003 via a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display 1011 may also be connected to the bus 1013 via an interface, such as a display adapter 1009. It is contemplated that the computing device 1001 may have more than one display adapter 1009 and the computing device 1001 may have more than one display 1011. A display 1011 may be a monitor, an LCD (Liquid Crystal Display), light emitting diode (LED) display, television, smart lens, smart glass, and/or a projector. In addition to the display 1011, other output peripheral devices may comprise components such as speakers (not shown) and a printer (not shown) which may be connected to the computing device 1001 via Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made via a network 1015, such as a local area network (LAN) and/or a general wide area network (WAN). Such network connections may be through a network adapter 1008. A network adapter 1008 may be implemented in both wired and wireless environments. Such networking environments are conventional and commonplace in dwellings, offices, enterprise-wide computer networks, intranets, and the Internet. In further exemplary aspects, it is contemplated that the computing device 1001 can be in communication with the remote computing devices 1014a,b,c through a Cloud-based network.

Application programs and other executable program components such as the operating system 1005 are shown herein as discrete blocks, although it is recognized that such programs and components may reside at various times in different storage components of the computing device 1001, and are executed by the one or more processors 1003 of the computing device 1001. An implementation of the wheelchair tracking software 1006 may be stored on or sent across some form of computer readable media. Any of the disclosed methods may be performed by processor-executable instructions embodied on computer readable media.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A disinfection system for a wheelchair, the disinfection system comprising:
an enclosure configured to receive the wheelchair therein; and
at least one disinfection lamp disposed within the enclosure,
wherein the enclosure is configured to receive the wheelchair therein.

Aspect 2: The disinfection system of aspect 1, wherein the enclosure comprises a pair of sidewalls that are spaced along a horizontal axis, wherein the pair of sidewalls are movably coupled to each other about and between a retracted configuration and an expanded configuration.

Aspect 3: The disinfection system of aspect 1, wherein the enclosure comprises a front wall, a rear wall, a top wall extending between a top edge of the front wall and a top edge of the rear wall, and an entry door pivotably secured to a first side edge of one of the front wall and the rear wall.

Aspect 4: The disinfection system of aspect 3, wherein the enclosure further comprises an exit door pivotably secured to a second side edge of the one of the front wall or the rear wall.

Aspect 5: The disinfection system of aspect 4, wherein the entry door and the exit door are both pivotably secured to the rear wall of the enclosure.

Aspect 6: The disinfection system of aspect 5, wherein the at least one disinfection lamp comprises a plurality of disinfection lamps, and wherein at least one disinfection lamp of the plurality of disinfection lamps is disposed on an inside surface of each of the front wall, the rear wall, and the top wall.

Aspect 7: The disinfection system of aspect 6, wherein disinfection lamps of the plurality of disinfection lamps are disposed on an inside surface of both the entry door and the exit door.

Aspect 8: The disinfection system of aspect 7, further comprising a pair of wheels disposed adjacent a bottom edge of the rear wall, and a handle assembly disposed adjacent the top edge of the rear wall.

Aspect 9: The disinfection system of aspect 7, further comprising a motion sensor disposed inside the enclosure, the motion sensor being configured to secure power to the plurality of disinfection lamps when motion of an object is detected inside the enclosure.

Aspect 10: The disinfection system of aspect 4, further comprising a first contact sensor and a second contact sensor that are configured to detect when the entry door and the exit door are in an open position, respectively, thereby securing power to the plurality of disinfection lamps when one or both the entry door and the exit door are in the open position.

Aspect 11: The disinfection system of aspect 4, wherein the bottom of the enclosure is open and defined by a bottom edge of each of the front wall, the rear wall, the entry door, and the exit door.

Aspect 12: The disinfection system of aspect 3, wherein the enclosure further comprises a side wall extending downwardly from a side edge of the top wall and between side edges of the front wall and the rear wall.

Aspect 13: The disinfection system of aspect 12, further comprising a housing extending downwardly from an inside surface of the top wall of the enclosure, the housing comprising:
a front wall spaced from an inside surface of the entry door, defining a gap therebetween;
a first side wall spaced from an inside surface of the rear wall of the enclosure, defining a gap therebetween; and
a second side wall spaced from an inside surface of the front wall of the enclosure, defining a gap therebetween;
wherein the front wall of the housing extends between a front edge of the first side wall and a front edge of the second side wall, and
wherein the at least one disinfecting lamp comprises a plurality of disinfecting lamps, and wherein at least one disinfecting lamp of the plurality of disinfecting lamps is disposed on each of the front wall, the first side wall, and the second side wall of the housing.

Aspect 14: The disinfection system of aspect 13, wherein the housing further comprises a bottom wall that extends between a bottom edge of the first side wall and a bottom edge of the second wall, and wherein at least one disinfection lamp of the plurality of disinfection lamps is disposed on the bottom wall of the housing.

Aspect 15: The disinfection system of aspect 4, wherein the front wall, rear wall, top wall, entry door, and exit door are all constructed of one of aluminum and stainless steel.

Aspect 16: The disinfection system of aspect 4, further comprising a corral area disposed in proximity to the exit door of the enclosure, wherein the corral area is configured to receive a plurality of wheelchairs therein.

Aspect 17: The disinfection system of aspect 3, wherein the at least one disinfection lamp comprises at least one ultraviolet-C lamp.

Aspect 18: A wheelchair tracking system comprising:
a plurality of enclosures configured to receive a wheelchair;
at least one wheelchair, wherein each wheelchair is received within a respective enclosure of the plurality of enclosures; and
at least one computing device configured to:
cause one enclosure of the plurality of enclosures having a wheelchair therein to release the wheelchair upon receipt of an identifier of a user.

Aspect 19: The wheelchair tracking system of aspect 1, wherein each enclosure of the plurality of enclosures further comprises at least one disinfection lamp therein.

Aspect 20: The wheelchair tracking system of aspect 1, wherein the at least one computing device is configured to contact the user after a predetermined time if the wheelchair is not returned to an enclosure of the plurality of enclosures.

Aspect 21: A disinfection system for a wheelchair, comprising:
an enclosure defining an interior cavity, the enclosure including a front wall, a rear wall, a top wall extending between a top edge of the front wall and a top edge of the rear wall, and an entry door pivotably secured to a first side edge of one of the front wall and the rear wall; and
an ultraviolet-C light source disposed within the interior cavity of the enclosure,
wherein the enclosure is configured to receive the wheelchair within the interview cavity.

Aspect 22: The disinfection system of aspect 21, wherein the enclosure further comprises an exit door pivotably secured to a second side edge of the one of the front wall and the rear wall.

Aspect 23: The disinfection system of aspect 21, wherein ultraviolet-C light source comprises at least one disinfection lamp.

Aspect 24: The disinfection system of aspect 21, further comprising a motion sensor disposed inside the interior cavity of the enclosure, the motion sensor being configured to stop delivery of power to the ultraviolet-C light source when motion of an object is detected inside the enclosure.

Aspect 25: The disinfection system of aspect 21, further comprising a contact sensor that is configured to detect when the entry door is in an open position, thereby stopping delivery of power to the source of ultraviolet-C light when the entry door is in the open position.

Aspect 26: The disinfection system of aspect 21, wherein the bottom of the enclosure is open.

Aspect 27: The disinfection system of aspect 21, further comprising a housing extending downwardly from an inside surface of the top wall of the enclosure, the housing comprising:
a first side wall spaced from an inside surface of the rear wall of the enclosure, defining a gap therebetween;
a second side wall spaced from an inside surface of the front wall of the enclosure, defining a gap therebetween; and
a front wall extending between a front edge of the first side wall and a front edge of the second side wall, the front wall being spaced from an inside surface of the entry door and defining a gap therebetween; and
wherein the ultraviolet-C light source rather comprises a plurality of distributing lamps, and
wherein disinfecting lamps of the plurality of disinfecting lamps are disposed on the front wall, the first side wall, and the second side wall of the housing.

Aspect 28: The disinfection system of aspect 27, wherein the housing further comprises a bottom wall that extends between a bottom edge of the first side wall and a bottom edge of the second wall, and wherein disinfection lamps of the plurality of disinfection lamps are disposed on the bottom wall of the housing.

Aspect 29: The disinfection system of aspect 22, wherein the front wall, rear wall, top wall, entry door, and exit door are all constructed of one of aluminum and stainless steel.

Although the foregoing invention has been described in some detail by way of illustration and example for purposes of clarity of understanding, certain changes and modifications may be practiced within the scope of the appended claims.

What is claimed is:

1. A disinfection system for a wheelchair, the disinfection system comprising:
an enclosure configured to receive the wheelchair therein; and
at least one disinfection lamp disposed within the enclosure,
wherein the enclosure is configured to receive the wheelchair therein.

2. The disinfection system of claim 1, wherein the enclosure comprises a pair of sidewalls that are spaced along a horizontal axis, wherein the pair of sidewalls are movably coupled to each other about and between a retracted configuration and an expanded configuration.

3. The disinfection system of claim 1, wherein the enclosure comprises a front wall, a rear wall, a top wall extending between the front wall and the rear wall, and an entry door pivotably secured to one of the front wall and the rear wall.

4. The disinfection system of claim 3, wherein the enclosure further comprises an exit door pivotably secured to the one of the front wall or the rear wall.

5. The disinfection system of claim 4, wherein the entry door and the exit door are both pivotably secured to the rear wall of the enclosure.

6. The disinfection system of claim 5, wherein the at least one disinfection lamp comprises a plurality of disinfection lamps, and wherein at least one disinfection lamp of the plurality of disinfection lamps is disposed on an inside surface of each of the front wall, the rear wall, and the top wall.

7. The disinfection system of claim 6, wherein disinfection lamps of the plurality of disinfection lamps are disposed on an inside surface of both the entry door and the exit door.

8. The disinfection system of claim 7, further comprising at least a pair of wheels coupled to the enclosure, and a handle assembly coupled to the enclosure.

9. The disinfection system of claim 7, further comprising a motion sensor disposed inside the enclosure, the motion sensor being configured to secure power to the plurality of disinfection lamps when motion of an object is detected inside the enclosure.

10. The disinfection system of claim 4, further comprising a first contact sensor and a second contact sensor that are configured to detect when the entry door and the exit door are in an open position, respectively, thereby securing power to the plurality of disinfection lamps when one or both the entry door and the exit door are in the open position.

11. The disinfection system of claim 4, wherein the bottom of the enclosure is open and defined by bottom edges of each of the front wall, the rear wall, the entry door, and the exit door.

12. The disinfection system of claim 3, wherein the enclosure further comprises a side wall extending downwardly from the top wall and between the front wall and the rear wall.

13. The disinfection system of claim 12, further comprising a housing extending downwardly from an inside surface of the top wall of the enclosure, the housing comprising:
a front wall spaced from an inside surface of the entry door, defining a gap therebetween;
a first side wall spaced from an inside surface of the rear wall of the enclosure, defining a gap therebetween; and
a second side wall spaced from an inside surface of the front wall of the enclosure, defining a gap therebetween;
wherein the front wall of the housing extends between the first side wall and the second side wall, and
wherein the at least one disinfecting lamp comprises a plurality of disinfecting lamps, and wherein at least one disinfecting lamp of the plurality of disinfecting lamps is disposed on each of the front wall, the first side wall, and the second side wall of the housing.

14. The disinfection system of claim 13, wherein the housing further comprises a bottom wall that extends between the first side wall and the second wall, and wherein at least one disinfection lamp of the plurality of disinfection lamps is disposed on the bottom wall of the housing.

15. The disinfection system of claim 4, wherein the front wall, rear wall, top wall, entry door, and exit door are all constructed of one of aluminum and stainless steel.

16. The disinfection system of claim 4, further comprising a corral area disposed in proximity to the exit door of the enclosure, wherein the corral area is configured to receive a plurality of wheelchairs therein.

17. The disinfection system of claim 1, wherein the at least one disinfection lamp comprises at least one ultraviolet-C lamp.

18. A wheelchair tracking system comprising:
a plurality of enclosures configured to receive a wheelchair;
at least one wheelchair, wherein each wheelchair is received within a respective enclosure of the plurality of enclosures; and
at least one computing device configured to:
cause one enclosure of the plurality of enclosures having a wheelchair therein to release the wheelchair upon receipt of an identifier of a user.

19. The wheelchair tracking system of claim 18, wherein each enclosure of the plurality of enclosures further comprises at least one disinfection lamp therein.

20. The wheelchair tracking system of claim 18, wherein the at least one computing device is configured to contact the user after a predetermined time if the wheelchair is not returned to an enclosure of the plurality of enclosures.
